(19) United States
(12) Patent Application Publication
(10) Pub. No.: US 2026/0020992 A1
(43) Pub. Date: Jan. 22, 2026

(54) HYGIENE ARTICLES CONTAINING NANOFIBERS

(71) Applicants: PROFIT ROYAL PHARMACEUTICAL LIMITED, Hong Kong (CN); JST POWER EQUIPMENT INC., Lake Mary, FL (US)

(72) Inventors: Hang Ching KWOK, Hong Kong (CN); Ho Wang TONG, Hong Kong (CN); Chak Wing YIU, Hong Kong (CN); Zhicong ZHANG, Hong Kong (CN)

(21) Appl. No.: 18/997,215

(22) PCT Filed: May 29, 2023
(86) PCT No.: PCT/CN2023/096743

§ 371 Date: Jan. 21, 2025

Related U.S. Application Data
(60) Provisional application No. 63/369,264, filed on Jul. 25, 2022.

(51) Int. Cl. A61F 13/511; A61F 13/15; A61F 13/51; A61F 13/512; A61F 13/514; A61F 13/53; A61F 13/531; A61L 15/18; A61L 15/24; A61L 15/42; A61L 15/44

(52) U.S. Cl. CPC A61F 13/51113; A61F 13/51121; A61F 13/5116; A61F 13/5126; A61F 13/51401; A61F 13/531; A61L 15/18; A61L 15/24; A61L 15/425; A61L 15/44; A61F 2013/15422; A61F 2013/51004; A61F 2013/51023; A61F 2013/51117; A61F 2013/5127; A61F 2013/530481

(57) ABSTRACT

Disposable hygiene article including a top sheet comprising a perforated hydrophilic non-woven fabric layer, wherein the perforated hydrophilic non-woven fabric layer has a plurality of pores and a skin facing surface and a garment facing surface opposite the skin facing surface; and a nanofiber layer disposed on the garment facing surface of the perforated hydrophilic non-woven fabric layer, wherein the nanofiber layer has densely packed nanofibers disposed between at least a portion of the plurality of pores; and loosely packed nanofibers disposed over at least a portion of the plurality of pores.

### CROSS-REFERENCE TO RELATED APPLICATIONS

The present application claims priority from U.S. Provisional Patent Application No. 63/369,264, filed on Jul. 25, 2022, which is hereby incorporated by reference in its entirety.

### TECHNICAL FIELD

The present disclosure relates to disposable hygiene articles. More particularly, the present disclosure relates to disposable hygiene articles comprising a nanofiber layer capable of controlled release of active ingredients to the wearer's skin and improved fluid retention and redistribution.

### BACKGROUND

Disposable hygiene articles, such as diapers, training pants, incontinence garments, adult incontinence pads, and feminine care products, are typically worn close to a wearer's skin to absorb discharged bodily fluids that can expose the wearer's skin to biological and physical irritants. Prolonged exposure to such irritants can result in dermatitis, inflammation, and wearer discomfort.

Disposable hygiene articles containing highly efficient absorbent cores configured to rapidly transfer fluids from the body of the wearer to an absorbent core have been developed that partially address the problem of skin irritation and wearer comfort. However, prolonged use of such disposable hygiene articles can still lead to skin irritation caused by friction between the skin and non-woven materials typically used in their construction.

Disposable hygiene articles configured to release active ingredients that can treat and/or prevent skin irritation and related complications have been developed. However, such disposable hygiene articles can experience burst release of the active ingredients upon exposure to bodily fluids.

Accordingly, there exists a need for improved disposable hygiene articles that overcome at least some of the disadvantages described above.

### SUMMARY

The present disclosure is generally related to disposable hygiene articles with improved body fluid redistribution and retention and controlled release of active ingredients useful for calming the skin, protecting the skin from irritation or infection, and/or promoting healing of rashes/broken skin.

In a first aspect, provided herein is a disposable hygiene article comprising: a top sheet comprising a perforated hydrophilic non-woven fabric layer, wherein the perforated hydrophilic non-woven fabric layer comprises a plurality of pores and a skin facing surface and a garment facing surface opposite the skin facing surface; and a nanofiber layer disposed on the garment facing surface of the perforated hydrophilic non-woven fabric layer, wherein the nanofiber layer comprises densely packed nanofibers disposed between at least a portion of the plurality of pores; and loosely packed nanofibers disposed over at least a portion of the plurality of pores, wherein the densely packed nanofibers are present at a density of 20-30 nanofibers per 100 μm³ and the loosely packed nanofibers are present at a density of 5-10 nanofibers per 100 μm³.

In certain embodiments, the densely packed nanofibers and the loosely packed nanofibers have an average diameter of 100 to 1,000 nm.

In certain embodiments, each of the plurality of pores has a diameter of 100-1,000 μm.

In certain embodiments, the plurality of pores has a pore-to-pore distance of 100-1,000 μm.

In certain embodiments, the densely packed nanofibers and the loosely packed nanofibers comprise a polymer selected from the group consisting of polymers comprising cellulose acetate (CA), polyamide 6 (PA6), polystyrene (PS), polyacrylonitrile (PAN), polyacrylonitrile co-polymer with methyl acrylate (n-PAN), polyvinylpyrrolidone (PVP), polyvinyl alcohol (PVA), poly(lactic acid) (PLA), poly(lactic-co-glycolic acid) (PLGA), polybutylene terephthalate (PBT), polyurethane (PU), gelatin, chitosan, polyhydroxybutyrate-co-hydroxyvalerate (PHBV), and combinations thereof.

In certain embodiments, the perforated hydrophilic non-woven fabric layer comprises microfibers having an average diameter of 10 to 30 μm.

In certain embodiments, the perforated hydrophilic non-woven fabric layer comprises polyethylene, polypropylene, or combinations thereof.

In certain embodiments, the nanofiber layer further comprises an active ingredient selected from the group consisting of an antioxidant, an anti-inflammatory, an anti-microbial, an emollient, and mixtures thereof.

In certain embodiments, the active ingredient is selected from the group consisting of calamine, dimethicone, kaolin, lanolin, petrolatum, talc, cornstarch, white petrolatum, zinc oxide, silver, copper, copper oxide, titanium oxide, iodine, triclosan, polyethylene glycol (PEG), sodium alginate, and mixtures thereof.

In certain embodiments, the microfibers have an average diameter of 10 to 30 μm; the densely packed nanofibers and the loosely packed nanofibers have an average diameter of 100 to 1,000 μm; the plurality of pores has a pore-to-pore distance of 100-1,000 μm; the densely packed nanofibers and the loosely packed nanofibers comprise a polymer selected from the group consisting of polymers comprising cellulose acetate (CA), polyamide 6 (PA6), polystyrene (PS), polyacrylonitrile (PAN), polyacrylonitrile co-polymer with methyl acrylate (n-PAN), polyvinylpyrrolidone (PVP), polyvinyl alcohol (PVA), poly(lactic acid) (PLA), poly(lactic-co-glycolic acid) (PLGA), polybutylene terephthalate (PBT), polyurethane (PU), gelatin, chitosan, polyhydroxybutyrate-co-hydroxyvalerate (PHBV), and combinations thereof; and the top sheet comprises non-woven material comprising fibers comprising polyethylene, polypropylene, or combinations thereof.

In certain embodiments, the nanofiber layer further comprises an active ingredient selected from the group consisting of antioxidant, an anti-inflammatory, an anti-microbial, an emollient, and mixtures thereof.

In certain embodiments, the disposable hygiene article of the first aspect further comprises: a back sheet; and an absorbent core disposed between the back sheet and the nanofiber layer, wherein the absorbent core comprises a superabsorbent polymer (SAP).

In certain embodiments, the back sheet comprises polypropylene, polyethylene, nylon, polyester, or a combination thereof.

In certain embodiments, the SAP comprises sodium polyacrylate.

In certain embodiments, the microfibers have an average diameter of 10 to 30 μm; the densely packed nanofibers and the loosely packed nanofibers have an average diameter of 100 to 1,000 nm; the plurality of pores has a pore-to-pore distance of 100-1,000 μm; the densely packed nanofibers and the loosely packed nanofibers comprise a polymer selected from the group consisting of polymers comprising cellulose acetate (CA), polyamide 6 (PA6), polystyrene (PS), polyacrylonitrile (PAN), polyacrylonitrile co-polymer with methyl acrylate (n-PAN), polyvinylpyrrolidone (PVP), polyvinyl alcohol (PVA), poly(lactic acid) (PLA), poly(lactic-co-glycolic acid) (PLGA), polybutylene terephthalate (PBT), polyurethane (PU), gelatin, chitosan, polyhydroxybutyrate-co-hydroxyvalerate (PHBV), and combinations thereof; and the top sheet comprises non-woven material comprising fibers comprising polyethylene, polypropylene, or combinations thereof.

In certain embodiments, the nanofiber layer further comprises an active ingredient selected from the group consisting of an antioxidant, an anti-inflammatory, an anti-microbial, an emollient, and mixtures thereof.

In certain embodiments, the active ingredient is selected from the group consisting of calamine, dimethicone, kaolin, lanolin, petrolatum, talc, cornstarch, white petrolatum, zinc oxide, silver, copper, copper oxide, titanium oxide, iodine, triclosan, polyethylene glycol (PEG), sodium alginate, and mixtures thereof.

In a second aspect, provided herein is a diaper comprising the disposable hygiene article of the first aspect.

In a third aspect, provided herein is a diaper comprising the disposable hygiene article of any embodiment described herein.

### BRIEF DESCRIPTION OF THE DRAWINGS

The above and other objects and features of the present disclosure will become apparent from the following description of the disclosure, when taken in conjunction with the accompanying drawings.

FIG. 1 depicts a schematic view of a disposable hygiene article in accordance with certain embodiments described herein.

FIG. 2 depicts scanning electron microscopy (SEM) images of a top sheet in a disposable hygiene article.

FIG. 3 depicts SEM images of densely packed nanofibers and loosely packed nanofibers disposed on a perforated hydrophilic non-woven fabric of the top sheet.

FIG. 4 depicts SEM images of densely packed nanofibers and loosely packed nanofibers disposed on a pore in the perforated hydrophilic non-woven fabric of the top sheet.

FIG. 5 depicts a schematic view of a disposable hygiene article in accordance with certain embodiments described herein.

FIG. 6 depicts a sectional view of a disposable hygiene article in accordance with certain embodiments described herein.

FIG. 7 depicts a view showing advantages of the disposable hygiene article in accordance with certain embodiments described herein compared to conventional hygiene articles.

FIG. 8 depicts a schematic view of nanofiber layer and a perforated hydrophilic non-woven fabric layer in accordance with certain embodiments described herein.

FIG. 9 depicts a schematic view of an exemplary setup for manufacturing the top sheet by electrospinning in accordance with certain embodiments described herein.

FIG. 10 depicts a graph showing the results of controlled release experiments of active ingredients from the nanofiber layer in accordance with certain embodiments described herein.

### DETAILED DESCRIPTION

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

It should be understood that the order of steps or order for performing certain actions is immaterial so long as the present teachings remain operable. Moreover, two or more steps or actions may be conducted simultaneously.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10%, ±7%, ±5%, ±3%, ±1%, or +0% variation from the nominal value unless otherwise indicated or inferred.

The term “skin facing surface” and “skin facing side” refer to surfaces of hygiene articles and/or components thereof which face a wearer's skin when the hygiene articles are worn, and the term “garment facing surface” and “garment facing side” refer to surfaces of absorbent articles and/or components thereof that face away from a wearer's skin when the absorbent articles are worn. Hygiene articles and components thereof, including the top sheet, nanofiber layer, back sheet, absorbent core, and any individual materials of their components, have a skin facing surface and/or side and a garment facing surface and/or side.

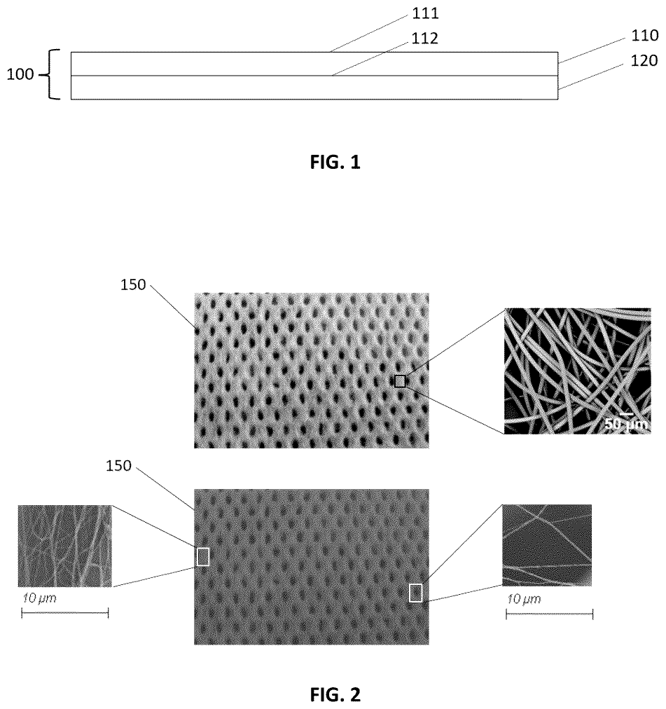

Referring to FIG. 1, the present disclosure provides a disposable hygiene article 100 comprising: a top sheet 110 comprising a perforated hydrophilic non-woven fabric layer, wherein the perforated hydrophilic non-woven fabric layer comprises a plurality of pores and a skin facing surface 111 and a garment facing surface 112 opposite the skin facing surface; and a nanofiber layer 120 disposed on the garment facing surface of the perforated hydrophilic non-woven fabric layer, wherein the nanofiber layer 120 comprises densely packed nanofibers disposed between at least a portion of the plurality of pores; and loosely packed nanofibers disposed over at least a portion of the plurality of pores, wherein the densely packed nanofibers are present at a density of 20-30 nanofibers per 100 μm³ and the loosely packed nanofibers are present at a density of 5-10 nanofibers per 100 μm³.

The perforated hydrophilic non-woven fabric layer can be made of any suitable relatively liquid-pervious material known in the art that permits passage of liquid therethrough. Examples of suitable perforated hydrophilic non-woven fabric layer materials include non-woven spunbond or carded webs of polypropylene, polyethylene, nylon, polyester and blends of these materials.

In instances in which the perforated hydrophilic non-woven fabric layer is prepared from a hydrophobic polymer, such as polypropylene, or a perforated hydrophobic non-woven fabric layer comprising a hydrophobic polymer, the hydrophobic polymer or the surface of the perforated hydrophobic non-woven fabric layer can be treated, such as by plasma treatment, to increase the hydrophilicity of the polymer or surface of the perforated hydrophobic non-woven fabric layer. Apart from plasma treatment, hydrophilic modification of the non-woven fabric layer can be accomplished by modification on fabric preparation process or modification of the hydrophobic polymer fabric surface by thin layer deposition, graft polymerization, physical modification, and combinations thereof.

In certain embodiments, the perforated hydrophilic non-woven fabric layer comprises plasma treated polypropylene to increase surface hydrophilization.

The perforated hydrophilic non-woven fabric layer comprises microfibers having an average diameter of 10 to 30 μm, 12 to 28 μm, 14 to 26 μm, 16 to 24 μm, 18 to 24 μm, 18 to 22 μm, 20 to 22 μm, 10 to 25 μm, 10 to 20 μm, 10 to 15 μm, 15 to 30 μm, 20 to 30 μm, or 25 to 30 μm. In certain embodiments, the perforated hydrophilic non-woven fabric layer comprises microfibers having an average diameter of about 20 μm.

Each of the plurality of pores present in the perforated hydrophilic non-woven fabric layer can have a diameter of 100-1,000 μm, 200-900 μm, 300-800 μm, 400-700 μm, 500-600 μm, 100-900 μm, 100-800 μm, 100-700 μm, 100-600 μm, 100-500 μm, 200-1,000 μm, 300-1,000 μm, 400-1,000 μm, 500-1,000 μm, 200-900 μm, 300-800 μm, 400-700 μm, 400-600 μm, or 450-550 μm. In certain embodiments, each of the plurality of pores present in the perforated hydrophilic non-woven fabric layer is about 500 μm.

The pore-to-pore distance of the perforated hydrophilic non-woven fabric layer can be 100-1,000 μm, 200-900 μm, 300-800 μm, 400-700 μm, 500-600 μm, 100-900 μm, 100-800 μm, 100-700 μm, 100-600 μm, 100-500 μm, 200-1,000 μm, 300-1,000 μm, 400-1,000 μm, 500-1,000 μm, 200-900 μm, 300-800 μm, 400-700 μm, 400-600 μm, or 450-550 μm. In certain embodiments, pore-to-pore distance of the perforated hydrophilic non-woven fabric layer is about 500 μm.

The densely packed nanofibers and the loosely packed nanofibers in the nanofiber layer can comprise one or more polymer selected from the group consisting of polymers comprising cellulose acetate (CA), polyamide 6 (PA6), polystyrene (PS), polyacrylonitrile (PAN), polyacrylonitrile co-polymer with methyl acrylate (n-PAN), polyvinylpyrrolidone (PVP), polyvinyl alcohol (PVA), poly(lactic acid) (PLA), poly(lactic-co-glycolic acid) (PLGA), polybutylene terephthalate (PBT), polyurethane (PU), gelatin, chitosan, polyhydroxybutyrate-co-hydroxyvalerate (PHBV), and blends and copolymers thereof. In certain embodiments, the nanofiber layer can comprise one or more polymer selected from the group consisting of polymers comprising PA6, PAN, n-PAN, and blends and copolymers thereof.

The densely packed nanofibers and the loosely packed nanofibers in the nanofiber layer can have a diameter of 100 to 1,000 nm. In certain embodiments, the densely packed nanofibers and the loosely packed nanofibers in the nanofiber layer have a diameter of 200-900 nm, 300-900 nm, 400-900 nm, 500-900 nm, 600-900 nm, 700-900 nm, 800-900 nm, 200-800 nm, 300-800 nm, 400-700 nm, 500-600 nm, 100-300 nm, or 700-900 nm. In certain embodiments, the densely packed nanofibers and the loosely packed nanofibers in the nanofiber layer have a diameter of about 200 nm or about 800 nm.

The densely packed nanofibers can be present at a density of 20-30, 21-29, 22-28, 23-27, 24-26, 20-25, or 25-30 nanofibers per 100 μm³. In certain embodiments, the densely packed nanofibers can be present at a density of about 20 or about 30 nanofibers per 100 μm³. The loosely packed nanofibers can be present at a density of 5-10, 6-9, 7-8, 5-7, or 7-10 nanofibers per 100 μm³. In certain embodiments, the loosely packed nanofibers can be present at a density of about 5 to about 10 nanofibers per 100 μm³.

In certain embodiments, the nanofiber layer disposed on the garment facing surface of the perforated hydrophilic non-woven fabric layer has a thickness between 0.7-0.8 mm.

The nanofiber layer disposed on the garment facing surface of the perforated hydrophilic non-woven fabric layer can have a basis weight of 20-100 g/m², 30-90 g/m², 40-80 g/m², 50-70 g/m², 20-50 g/m², or 50-100 g/m².

In certain embodiments, the nanofiber layer disposed on the garment facing surface of the perforated hydrophilic non-woven fabric layer exhibits a pressure drop of 0.5-10, 0.5-9, 0.5-8, 0.5-7, 0.5-6, 0.5-5, 0.5-4, 0.5-3, 0.5-2, or 0.5-1 mm H₂O when tested at a face velocity of 5.3 cm/s.

Advantageously, the nanofiber layer can further comprise an active ingredient that can calm the skin, protect the skin from irritation or infection, and/or promote healing of rashes/broken skin. In certain embodiments, the nanofiber layer further comprises an active ingredient selected from the group consisting of an antioxidant, an anti-inflammatory, an anti-microbial, an emollient, and mixtures thereof.

In certain embodiments, the ingredients are selected from the group consisting of calamine, dimethicone, kaolin, lanolin, petrolatum, talc, cornstarch, white petrolatum, zinc oxide, silver, copper, copper oxide, titanium oxide, iodine, triclosan, polyethylene glycol (PEG), sodium alginate, and mixtures thereof.

The active ingredient can be preset in the nanofiber layer at a concentration of 0.1-50 wt/wt % relative to the weight of the densely packed nanofiber polymer, the loosely packed nanofibers polymer, and the active ingredient. In certain embodiments, the active ingredient is preset in the nanofiber layer at a concentration of 5-50 wt/wt %, 10-50 wt/wt %, 15-50 wt/wt %, 20-50 wt/wt %, 25-50 wt/wt %, 30-50 wt/wt %, 35-50 wt/wt %, 40-50 wt/wt %, or 45-50 wt/wt % relative to the weight of the densely packed nanofiber polymer, the loosely packed nanofibers polymer, and the active ingredient. In certain embodiments, the active ingredient is preset in the nanofiber layer at a concentration of 14.2-33 wt/wt %, 14.2-29.4 wt/wt %, 16.67-29.4 wt/wt %, 14.2-16.67 wt/wt %, 16.67-33 wt/wt %, or 29.4-33 wt/wt % relative to the weight of the densely packed nanofiber polymer, the loosely packed nanofibers polymer, and the active ingredient.

The nanofiber layer is capable of both stabilizing and maintaining the properties of the active ingredient(s) while in the absence of moisture and releasing the active ingredient(s) in a controlled manner in the presence of moisture without burst release while redistributing the fluid across a greater area of the absorbent core resulting in improved absorption.

The nanofiber layer also increases the rate of fluid absorption and assists with redistributing fluids within the absorbent core, which can reduce rewetting.

FIG. 2 shows scanning electron microscopy (SEM) images of the top sheet 110. The top sheet 110 comprises a perforated hydrophilic non-woven fabric 150. The perforated hydrophilic non-woven fabric 150 comprises microfibers and nanofibers disposed on a surface of the hydrophilic non-woven fabric. Microfibers are visible in the SEM image with a scale bar of 50 μm. Nanofibers can be seen in the SEM images with a scale bar of 10 μm.

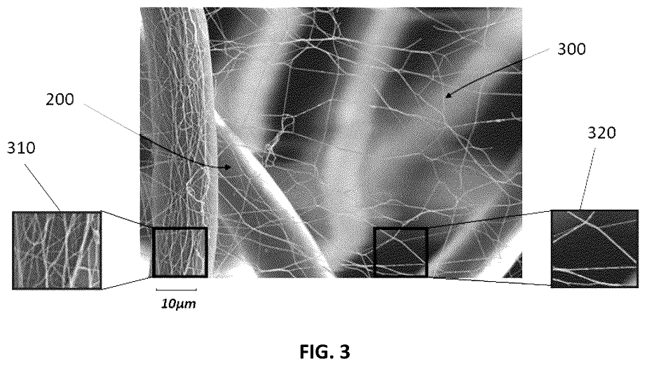

FIG. 3 shows an SEM image with a scale bar of 10 μm in which both microfibers and nanofibers are visible. The nanofibers 300 comprise densely packed nanofibers 310 disposed on the microfibers 200 and loosely packed nanofibers 320 disposed over perforations of the hydrophilic non-woven fabric.

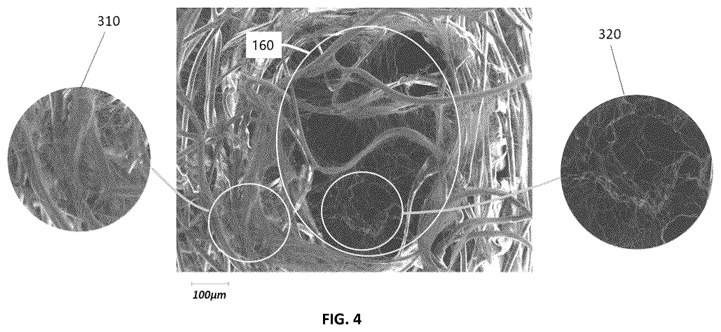

FIG. 4 shows an SEM image with a scale bar of 100 μm in which pores in the hydrophilic non-woven fabric layer are visible. The densely packed nanofibers 310 are disposed on the surface of the perforated hydrophilic non-woven fabric layer between at least a portion of the pores 160. The loosely packed nanofibers 320 are disposed over at least a portion of the plurality of pores 160.

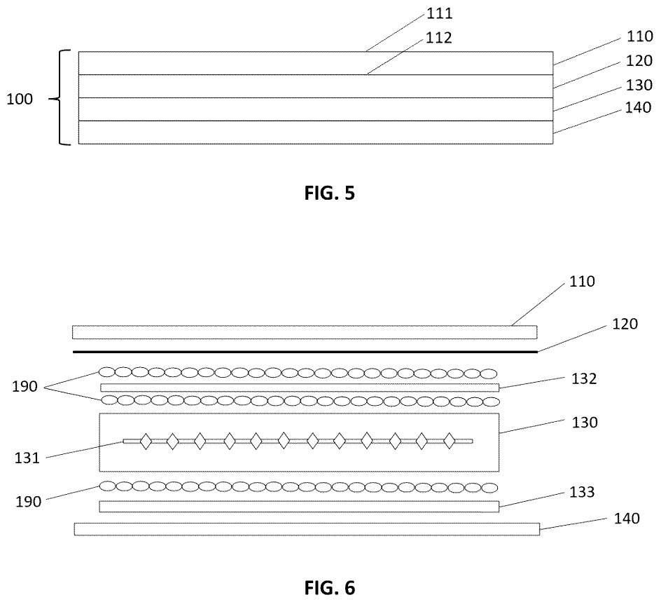

Referring to FIG. 5, in certain embodiments, the disposable hygiene article further comprises a back sheet 140; and an absorbent core 130 disposed between the back sheet 140 and the nanofiber layer 120.

The absorbent core 130, which is disposed between the nanofiber layer 120 and the back sheet 140, absorbs and retains bodily fluids that have penetrated the top sheet. The absorbent core may be any absorbent means which is capable of absorbing or retaining bodily liquids. The absorbent core may be manufactured in a wide variety of sizes and shapes (e.g., rectangular, oval, asymmetric, etc.) and from a wide variety of liquid-absorbent materials commonly used in absorbent articles, such as comminuted wood pulp. Examples of other suitable absorbent materials include, but are not limited to, creped cellulose wadding; meltblown polymers; chemically stiffened, modified or cross-linked cellulosic fibers; synthetic fibers such as crimped polyester fibers; peat moss; tissue including tissue wraps and tissue laminates; absorbent foams; and absorbent sponges. In certain embodiments, the absorbent core comprises non-woven fibrous material selected from the group consisting of polyester, polyethylene terephthalate, polyethylene, polypropylene, polyethylene terephthalate/polyethylene, polyethylene terephthalate/polypropylene, polypropylene/polyethylene, PLA, PLA/polypropylene, PVA, viscose, cotton, wool, acetate, polyvinylchloride, bamboo, polyacrylic acid/polyvinyl chloride, and copolymers, and blends thereof.

The absorbent core 130 can further comprise a SAP 131 (shown in FIG. 6). The type of SAP is not particularly limited. In certain embodiments, the SAP is a polyacrylate, a polymethacrylate, or mixtures and/or copolymers thereof. Exemplary, SAPs include, but are not limited to sodium polyacrylate, sodium polymethacrylate, potassium polyacrylate, potassium polymethacrylate, starch grafted polyacrylate, starch grafted polymethacrylate, polyvinyl alcohol grafted polyacrylate, polyvinyl alcohol grafted polymethacrylate, cellulose grafted polyacrylate, cellulose grafted polymethacrylate, and the like.

A variety of back sheet constructions and materials are available and known in the art, and the present disclosure is not intended to be limited to any specific materials or constructions of these components. The back sheet 140 may be made from any suitable pliable liquid-impervious material known in the art. Exemplary back sheet materials include, but are not limited to, films of polyethylene, polypropylene, polyester, nylon, and polyvinyl chloride and blends of these materials.

FIG. 6 is a sectional view of a disposable hygiene article in accordance with certain embodiments. Compared with FIG. 5, the disposable hygiene article further comprises an acquisition distribution layer (ADL) 132 disposed between the nanofiber layer 120 and the absorbent core 130. The ADL 132 can comprise a hydrophilic web that ensures faster passage of liquids into the absorbent core 130. The ADL 132 can comprise fibers selected from the group consisting of polyester, co-polyester, polypropylene, polyethylene, polylactic acid, polyamide, and copolymers, and mixtures thereof.

An adhesive means 190, such as a glue, can be used to affix the various components of the disposable hygiene articles described herein. For example, glue can be used to affix any one or more of the nanofiber layer 120, ADL 132, absorbent core 130, and back sheet 140 together. Optionally, a separate breathable sheet layer 133 can be disposed between the absorbent core 130 and the back sheet 140.

The disposable hygiene articles described herein can be manufactured in the configuration of wearable articles that are capable of absorbing large quantities of water and body fluids, such as urine, feces, menses, blood and other excretions. Such articles, include but are not limited to, diapers, training pants, incontinence garments, adult incontinence pads, feminine care sanitary napkins, feminine sanitary underwear, panty liners, maternity pads, disposable bed sheets, wound dressings, and the like.

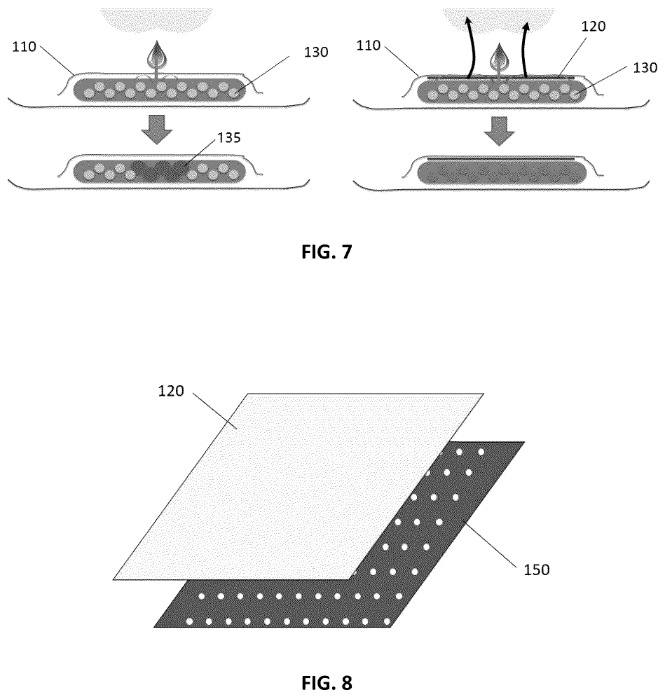

FIG. 7 shows the advantages achieved by the disposable hygiene article described herein (right) over the conventional hygiene articles (left). Conventional hygiene articles, such as diapers, are prone to rewet when a central portion 135 of the absorbent core 130 becomes saturated. This may slow down the absorption upon recurrent fluid addition. Nanofiber layer 120 disposed on the top sheet 110 increases the rate of fluid absorption and assists with redistributing fluids within the absorbent core 130, which can reduce rewetting. The nanofiber layer 120 may comprise active ingredients useful for calming the skin and protecting the skin from irritation or infection.

The disposable hygiene articles described herein can be manufactured using conventional methods well known to those of skill in the art.

The perforated hydrophilic non-woven fabric layer can be readily manufactured using well known methods. In certain embodiments, the perforated hydrophilic non-woven fabric layer is prepared by hot air bonding. In such instances hot air non-woven fabric is formed by the short fibers after carding, and the hot air on the drying equipment is used to penetrate the web, which is then heated and bonded.

Apart from hot air bonding, the perforated hydrophilic non-woven fabric layer can also be made by spunbonding. During spunbonding, after the polymer is extruded and stretched to form continuous filaments, the filaments are laid into a web, and the web is then subjected to self-adhesion, thermal bonding, chemical bonding, or mechanical reinforcement to make the web into a non-woven fabric.

The perforated hydrophilic non-woven fabric layer can then be loaded onto the electrospinning setup, such that the loosely packed nanofibers and the densely packed nanofibers can be deposited onto the perforated hydrophilic non-woven fabric layer during the electrospinning process.

FIG. 8 shows a schematic view of the nanofiber layer 120 and the perforated hydrophilic non-woven fabric layer 150. The nanofiber layer 120 can be deposited on the hydrophilic non-woven fabric layer 150 by using a free surface electrospinning process comprising: providing a solution comprising a polymer solution; applying an electric force to the polymer solution thereby forming nanofibers and depositing the nanofibers on the garment facing surface of the hydrophilic non-woven fabric layer thereby forming the nanofiber layer disposed on the garment facing surface of the perforated hydrophilic non-woven fabric layer.

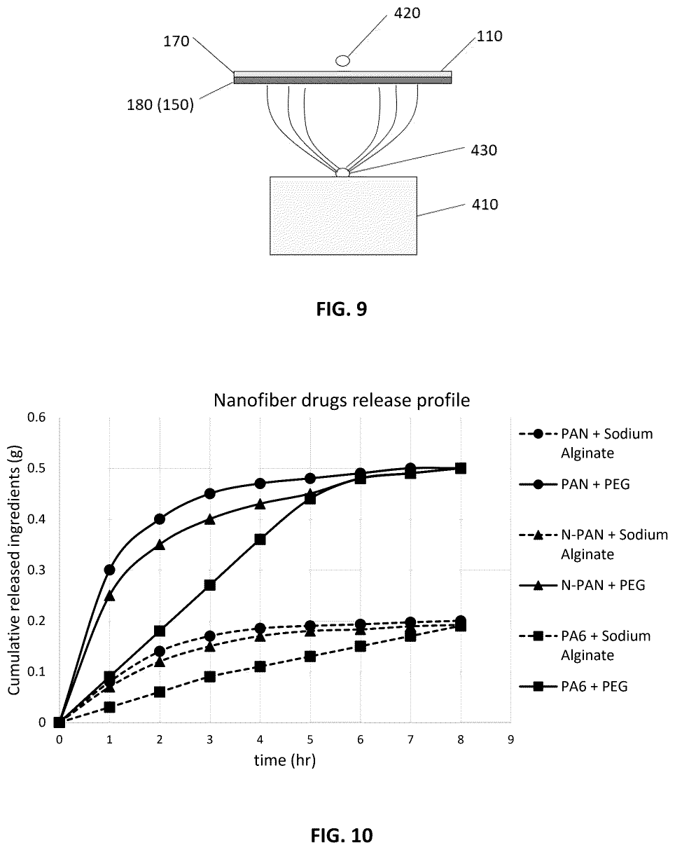

FIG. 9 shows a horizontal schematic view of the setup for manufacturing the top sheet 110 described herein by electrospinning in accordance with certain embodiments. The nanofibers can be electrospun from a polymer solution 410 optionally comprising one or more active ingredients. Unlike traditional electrospinning processes that produce one layer of substrate for nanofiber deposition, the methods described herein can be used to prepare the top sheet 110 comprising the perforated hydrophilic non-woven fabric layer 150 and the nanofiber layer 120 disposed on the garment facing surface of the perforated hydrophilic non-woven fabric layer 150. As shown in FIG. 9, the top sheet 110 comprises a top layer 170 and a bottom layer 180. The top layer 170 is proximal to the negatively charged collecting electrode 420. The top layer 170 is a non-woven fabric, which can have a sheet resistance of 1012 ohms per square. The bottom layer 180 is distal to the negatively charged collecting electrode 420. The bottom layer 180 can be the perforated hydrophilic non-woven fabric 150, which can have a sheet resistance of 109 ohms per square. The bottom layer 180 can be negatively charged by triboelectric process. Nanofibers can be deposited on the surface of the bottom layer during the electrospinning process.

During the electrospinning process, positive charges can be induced at the top layer 170 due to the presence of the negatively charged collecting electrode 420. During the electrospinning process, the polymer solution 410 is charged by the strong positive voltage provided by the positively charged spinning electrode 430. Taylor cones are generated on the positively charged spinning electrode 430, with the positive charges accumulated on the surface. When the electric force exerted on the Taylor cone overcomes the surface tension, polymer jets are drawn from the Taylor cone. After evaporation of solvent and solidification of the polymer jet, nanofibers are deposited onto the bottom layer 180. When nanofibers are approaching to the bottom layer 180, the nanofiber density is influenced by the charge distribution on the bottom layer 180. The top layer 170 with induced positive charges can repel most of the nanofibers through the pores in the bottom layer 180, resulting in loosely packed nanofibers over the pores. On the other hand, the negative charges between the pores of the bottom layer partially cancel out the positive charges in the top layer, resulting in minimal charge repulsion and hence densely packed nanofibers between the pores.

Performance Evaluation: Diapers with Nanofibers Vs Diapers without Nanofibers Four Features for Evaluation

The diapers comprising the nanofibers (specifically, the use of loosely packed nanofibers over the pores) and the corresponding diapers without nanofibers were evaluated, respectively, in terms of the following four features: (1) the absorption time at the first time of liquid addition, (2) the absorption time at the second time of liquid addition, (3) the rewet amount and (4) the leakage amount. The absorption time is defined as the time required for the diaper to absorb a specific amount of testing solution. The rewet amount is defined as the amount of testing solution returned back to the top sheet (proximal to the wearer's skin) of the diaper under a specific pressure after absorption of a specific amount of testing solution by the diaper. The leakage amount is defined as the amount of testing solution penetrated through the backing layer (distal to the wearer's skin) of the diaper under a specific pressure after absorption of a specific amount of testing solution by the diaper.

### EXAMPLES

### Example 1—Evaluation Procedure for Evaluating Absorption Time at the First Time of Liquid Addition, the Absorption Time at the Second Time of Liquid Addition, the Rewet Amount and the Leakage Amount

Diapers were evaluated according to the following procedure:

1. Put the diaper on a sample holder lined with absorbent paper (basis weight 140-150 g/m²; water absorption no less than 480%).

2. Place a dosing module onto the absorption area of the diaper surface.

3. Inject a certain volume (40 mL) of normal saline (0.9% w/v sodium chloride solution) into the dosing module under a certain pressure (1.8-2.2 kPa).

4. Record the time from the start of liquid addition to the complete absorption of normal saline by the diaper with the aid of an automatic timing device.

5. Add liquid to the same diaper twice and the absorption time of the diaper is represented by the absorption time at the first time of liquid addition and the absorption time at the second time of liquid addition, respectively.

6. Take out the dosage module after completion of the second liquid addition.

7. Place a certain number of layers of absorbent paper on the diaper.

8. Put the diaper into a pressure module under a certain pressure (3.8-4.2 kPa) for a specific time (1 minute).

9. The rewet amount of the diaper is expressed by the mass increase of the absorbent paper on the diaper.

10. The leakage amount of the diaper is expressed by the mass increase of the absorbent paper at the bottom of the diaper.

The absorption time at the first time of liquid addition, the absorption time at the second time of liquid addition, the rewet amount and the leakage amount of the diapers comprising the nanofibers (specifically, the use of loosely packed nanofibers over the pores) and the corresponding diapers without nanofibers are summarized in the table below. For all entries, the perforated hydrophilic non-woven fabric layer is prepared from plasma treated polypropylene.

| | | | | | Absorption | | |
|---|---|---|---|---|---|---|---|
| | | | | Absorption | time at the | | |
| | | | | time at the | second | | |
| | | | | first time | time of | | |
| | | | | of liquid | liquid | Rewet | Leakage |
| | | | | addition | addition | amount | amount |
| | | | | (s) | (s) | (g) | (g) |
| Without nanofibers | | | | 21.6 | 38.4 | 5.1 | 0.5 |
| Pore size of the top sheet: 500 μm | | | | | | | |
| Pore-to-pore distance: 500 μm | | | | | | | |
| Microfiber diameter of | | | | | | | |
| the top sheet: 20 μm | | | | | | | |
| With nanofibers | | | | | | | |
| | | Density | Density | | | | |
| | | of densely | of loosely | | | | |
| | | packed | packed | | | | |
| | | nano- | nano- | | | | |
| Polymer | | fibers | fibers | | | | |
| used for | Nano- | disposed | disposed | | | | |
| the | fiber | between | over | | | | |
| nano- | diameter | pores | pores | | | | |
| fibers | (nm) | (per μm³) | (per μm³) | | | | |
| PA6 | 200 | 20 | 5 | 15.6 | 28.3 | 0.2 | 0.3 |
| | 200 | 20 | 10 | 16.1 | 28.8 | 0.1 | 0.2 |
| | 200 | 30 | 5 | 15.7 | 28.1 | 0.2 | 0.3 |
| | 200 | 30 | 10 | 16.2 | 28.7 | 0.1 | 0.2 |
| | 800 | 20 | 5 | 15.9 | 28.8 | 0.2 | 0.3 |
| | 800 | 20 | 10 | 16.3 | 29.1 | 0.1 | 0.2 |
| | 800 | 30 | 5 | 16.1 | 29.2 | 0.2 | 0.3 |
| | 800 | 30 | 10 | 16.5 | 29.1 | 0.1 | 0.2 |
| PAN | 200 | 20 | 5 | 14.5 | 25.3 | 0.1 | 0.2 |
| | 200 | 20 | 10 | 15.1 | 25.9 | 0.1 | 0.1 |
| | 200 | 30 | 5 | 14.4 | 25.2 | 0.1 | 0.2 |
| | 200 | 30 | 10 | 15.3 | 25.7 | 0.1 | 0.1 |
| | 800 | 20 | 5 | 14.4 | 25.2 | 0.1 | 0.2 |
| | 800 | 20 | 10 | 15.2 | 26.1 | 0.1 | 0.1 |
| | 800 | 30 | 5 | 14.1 | 25.5 | 0.1 | 0.2 |
| | 800 | 30 | 10 | 15.2 | 25.8 | 0.1 | 0.1 |
| n-PAN | 200 | 20 | 5 | 12.3 | 21.6 | 0.2 | 0.2 |
| | 200 | 20 | 10 | 12.6 | 21.9 | 0.1 | 0.2 |
| | 200 | 30 | 5 | 12.2 | 21.7 | 0.1 | 0.2 |
| | 200 | 30 | 10 | 12.5 | 22.1 | 0.1 | 0.2 |
| | 800 | 20 | 5 | 12.1 | 21.5 | 0.1 | 0.2 |
| | 800 | 20 | 10 | 12.4 | 21.6 | 0.1 | 0.2 |
| | 800 | 30 | 5 | 12.3 | 21.8 | 0.1 | 0.2 |
| | 800 | 30 | 10 | 12.3 | 22.3 | 0.1 | 0.2 |

It can be seen from the results that the use of nanofibers can generally shorten the absorption time, reduce the rewet amount and reduce the leakage amount. The improvement highly depends on the nanofiber material. Among the three materials chosen, it can be seen from the table that n-PAN is the best polymer, followed by PAN and PA6, in terms of the improvement of the absorption time, rewet amount and leakage amount of the diaper.

### Example 2—Controlled Release of Active Ingredients Test Results

### Example 2A—Nanofibers Electrospun from a Solution with 10% n-PAN and 2% Sodium Alginate

Solution Preparation: 1 g of n-PAN was dissolved in 9 ml of N, N-dimethylformamide. The solution was mixed by a roller mixer at room temperature for over 8 hours until the n-PAN was completely dissolved in N,N-dimethylformamide. 0.2 g of sodium alginate was added into 1 ml of DI water and was stirred by a roller mixer under room temperature for over 8 hours. Two homogeneous solutions were mixed and were stirred by a vortex mixer for 1 minute until a stable emulsion was formed.

Electrospinning: The emulsion (i.e. the spinning solution) was filled in the carriage of the electrospinning equipment (NanoSpider, NS 1S500U, Elmarco, Czech). Before starting the electrospinning process, carriage was operated to ensure that electrode wire could be coated with the spinning solution. After starting the electrospinning process, voltage on the spinning electrode was set to +60 kV and voltage on the collecting electrode was set to −40 kV. The spinning solution was drawn into nanofibers that were deposited onto the surface of the bottom layer (i.e. the perforated hydrophilic non-woven fabric layer) of the composite substrate. After running out of the spinning solution, voltage was set to 0 for both electrodes (i.e. the spinning electrode and the collecting electrode).

Controlled Release Study: The release profile was investigated by High Performance Liquid Chromatography (HPLC). The cumulative active ingredient was studied hourly. The dotted curve with triangle dots in FIG. 10 shows the release profile of sodium alginate from the n-PAN nanofibers. Sodium alginate was gradually released from the n-PAN nanofibers within 8 hours. No burst release was found in the beginning and the release rate was reduced after 2 hours. Almost 100% of sodium alginate was released from the nanofibers after 8 hours.

### Example 2B—Nanofibers Electrospun from a Solution with 10% n-PAN and 5% PEG

Solution Preparation: 1 g of n-PAN was dissolved in 9 ml of N, N-dimethylformamide. The solution was mixed by a roller mixer at room temperature for over 8 hours until the n-PAN was completely dissolved in N,N-dimethylformamide. 0.5 g of PEG was added into 1 ml of DI water and was stirred by a roller mixer under room temperature for over 8 hours. Two homogeneous solutions were mixed and were stirred by a vortex mixer for 1 minute until a stable emulsion was formed.

Electrospinning: The emulsion (i.e. the spinning solution) was filled in the carriage of the electrospinning equipment (NanoSpider, NS 1S500U, Elmarco, Czech). Before starting the electrospinning process, carriage was operated to ensure that electrode wire could be coated with the spinning solution. After starting the electrospinning process, voltage on the spinning electrode was set to +60 kV and voltage on the collecting electrode was set to −40 kV. The spinning solution was drawn into nanofibers that were deposited onto the surface of the bottom layer (i.e. the perforated hydrophilic non-woven fabric layer) of the composite substrate. After running out of the spinning solution, voltage was set to 0 for both electrodes (i.e. the spinning electrode and the collecting electrode).

Controlled Release Study: The solid curve with triangle dots in FIG. 10 shows the release profile of PEG from the n-PAN nanofibers. Burst release of PEG was observed within the first 2 hours. PEG was gradually released from the n-PAN nanofibers after 2 hours. PEG was completely released from the nanofibers within 8 hours.

### Example 2C-Nanofibers Electrospun from a Solution with 10% PAN and 2% Sodium

### Alginate

Solution Preparation: 1 g of PAN was dissolved in 9 ml of N, N-dimethylformamide. The solution was mixed by a roller mixer at room temperature for over 8 hours until the PAN was completely dissolved in N,N-dimethylformamide. 0.2 g of sodium alginate was added into 1 ml of DI water and was stirred by a roller mixer under room temperature for over 8 hours. Two homogeneous solutions were mixed and were stirred by a vortex mixer for 1 minute until a stable emulsion was formed.

Electrospinning: The emulsion (i.e. the spinning solution) was filled in the carriage of the electrospinning equipment (NanoSpider, NS 1S500U, Elmarco, Czech). Before starting the electrospinning process, carriage was operated to ensure that electrode wire could be coated with the spinning solution. After starting the electrospinning process, voltage on the spinning electrode was set to +60 kV and voltage on the collecting electrode was set to −40 kV. The spinning solution was drawn into nanofibers that were deposited onto the surface of the bottom layer (i.e. the perforated hydrophilic non-woven fabric layer) of the composite substrate. After running out of the spinning solution, voltage was set to 0 for both electrodes (i.e. the spinning electrode and the collecting electrode).

Controlled Release Study: The dotted curve with circle dots in FIG. 10 shows the release profile of sodium alginate from the PAN nanofibers. PAN possessed a faster release rate of sodium alginate when compared with n-PAN. Most of the sodium alginate was released from PAN nanofibers within 4 hours and the rest was gradually released in the last 4 hours.

### Example 2D-Nanofibers Electrospun from a Solution with 10% PAN and 5% PEG

Solution Preparation: 1 g of PAN was dissolved in 9 ml of N, N-dimethylformamide. The solution was mixed by a roller mixer at room temperature for over 8 hours until the PAN was completely dissolved in N,N-dimethylformamide. 0.5 g of PEG was added into 1 ml of DI water and was stirred by a roller mixer under room temperature for over 8 hours. Two homogeneous solutions were mixed and were stirred by a vortex mixer for 1 minute until a stable emulsion was formed.

Electrospinning: The emulsion (i.e. the spinning solution) was filled in the carriage of the electrospinning equipment (NanoSpider, NS 1S500U, Elmarco, Czech). Before starting the electrospinning process, carriage was operated to ensure that electrode wire could be coated with the spinning solution. After starting the electrospinning process, voltage on the spinning electrode was set to +60 kV and voltage on the collecting electrode was set to −40 kV. The spinning solution was drawn into nanofibers that were deposited onto the surface of the bottom layer (i.e. the perforated hydrophilic non-woven fabric layer) of the composite substrate. After running out of the spinning solution, voltage was set to 0 for both electrodes (i.e. the spinning electrode and the collecting electrode).

Controlled Release Study: The solid curve with circle dots in FIG. 10 shows the release profile of PEG from the PAN nanofibers. PAN nanofibers still possessed faster release rate of PEG when compared with n-PAN nanofibers, indicating that the release profile of the same ingredient highly depends on the choice of the nanofiber materials. Burst release of PEG was observed within the first 2 hours, followed by a gradual release in the last 6 hours.

### Example 2E—Nanofibers Electrospun from a Solution with 12% PA6 and 2% Sodium Alginate

Solution Preparation: 1.2 g of PA6 was dissolved in 9 ml of a mixture of acetic acid (AA) and formic acid (FA). The ratio of AA:FA was 2:1. The solution was mixed by a roller mixer at 60° C. for over 8 hours until the PA6 was completely dissolved in the mixture of AA and FA. 0.2 g of sodium alginate was added into 1 ml of DI water and was stirred by a roller mixer under room temperature for over 8 hours. Two homogeneous solutions were mixed and were stirred by a vortex mixer for 1 minute until a stable emulsion was formed.

Electrospinning: The emulsion (i.e. the spinning solution) was filled in the carriage of the electrospinning equipment (NanoSpider, NS 1S500U, Elmarco, Czech). Before starting the electrospinning process, carriage was operated to ensure that electrode wire could be coated with the spinning solution. After starting the electrospinning process, voltage on the spinning electrode was set to +60 kV and voltage on the collecting electrode was set to −40 kV. The spinning solution was drawn into nanofibers that were deposited onto the surface of the bottom layer (i.e. the perforated hydrophilic non-woven fabric layer) of the composite substrate. After running out of the spinning solution, voltage was set to 0 for both electrodes (i.e. the spinning electrode and the collecting electrode).

Controlled Release Study: The dotted curve with square dots in FIG. 10 shows the release profile of sodium alginate from the PA6 nanofibers. The release rate of the PA6 nanofibers was stable when compared with n-PAN or PAN nanofibers. Sodium alginate was gradually released from the PA6 nanofibers within 8 hours and no burst release was observed.

### Example 2F—Nanofibers Electrospun from a Solution with 12% PA6 and 5% PEG

Solution Preparation: 1.2 g of PA6 was dissolved in 9 ml of a mixture of acetic acid (AA) and formic acid (FA). The ratio of AA:FA was 2:1. The solution was mixed by a roller mixer at 60° C. for over 8 hours until the PA6 was completely dissolved in the mixture of AA and FA. 0.5 g of PEG was added into 1 ml of DI water and was stirred by a roller mixer under room temperature for over 8 hours. Two homogeneous solutions were mixed and were stirred by a vortex mixer for 1 minute until a stable emulsion was formed.

Electrospinning: The emulsion (i.e. the spinning solution) was filled in the carriage of the electrospinning equipment (NanoSpider, NS 1S500U, Elmarco, Czech). Before starting the electrospinning process, carriage was operated to ensure that electrode wire could be coated with the spinning solution. After starting the electrospinning process, voltage on the spinning electrode was set to +60 kV and voltage on the collecting electrode was set to −40 kV. The spinning solution was drawn into nanofibers that were deposited onto the surface of the bottom layer (i.e. the perforated hydrophilic non-woven fabric layer) of the composite substrate. After running out of the spinning solution, voltage was set to 0 for both electrodes (i.e. the spinning electrode and the collecting electrode).

Controlled Release Study: The solid curve with square dots in FIG. 10 shows the release profile of PEG from the PA6 nanofibers. Most of the PEG was released from the PA6 nanofibers within 6 hours, followed by a gradual release in the last 2 hours. No burst release was observed and all PEG was released within 8 hours.

What is claimed is:

1. A disposable hygiene article comprising:
a top sheet comprising a perforated hydrophilic non-woven fabric layer, wherein the perforated hydrophilic non-woven fabric layer comprises a plurality of pores and a skin facing surface and a garment facing surface opposite the skin facing surface; and
a nanofiber layer disposed on the garment facing surface of the perforated hydrophilic non-woven fabric layer, wherein the nanofiber layer comprises densely packed nanofibers disposed between at least a portion of the plurality of pores; and loosely packed nanofibers disposed over at least a portion of the plurality of pores, wherein the densely packed nanofibers are present at a density of 20-30 nanofibers per 100 μm³ and the loosely packed nanofibers are present at a density of 5-10 nanofibers per 100 μm³.

2. The disposable hygiene article of claim 1, wherein the densely packed nanofibers and the loosely packed nanofibers have an average diameter of 100 to 1,000 nm.

3. The disposable hygiene article of claim 1, wherein each of the plurality of pores has a diameter of 100-1,000 μm.

4. The disposable hygiene article of claim 1, wherein the plurality of pores has a pore-to-pore distance of 100-1,000 μm.

5. The disposable hygiene article of claim 1, wherein the densely packed nanofibers and the loosely packed nanofibers comprise a polymer selected from the group consisting of polymers comprising cellulose acetate (CA), polyamide 6 (PA6), polystyrene (PS), polyacrylonitrile (PAN), polyacrylonitrile co-polymer with methyl acrylate (n-PAN), polyvinylpyrrolidone (PVP), polyvinyl alcohol (PVA), poly(lactic acid) (PLA), poly(lactic-co-glycolic acid) (PLGA), polybutylene terephthalate (PBT), polyurethane (PU), gelatin, chitosan, polyhydroxybutyrate-co-hydroxyvalerate (PHBV), and combinations thereof.

6. The disposable hygiene article of claim 1, wherein the perforated hydrophilic non-woven fabric layer comprises microfibers having an average diameter of 10 to 30 μm.

7. The disposable hygiene article of claim 1, wherein the perforated hydrophilic non-woven fabric layer comprises polyethylene, polypropylene, or combinations thereof.

8. The disposable hygiene article of claim 1, wherein the nanofiber layer further comprises an active ingredient selected from the group consisting of an antioxidant, an anti-inflammatory, an anti-microbial, an emollient, and mixtures thereof.

9. The disposable hygiene article of claim 8, wherein the active ingredient is selected from the group consisting of calamine, dimethicone, kaolin, lanolin, petrolatum, talc, cornstarch, white petrolatum, zinc oxide, silver, copper, copper oxide, titanium oxide, iodine, triclosan, polyethylene glycol (PEG), sodium alginate, and mixtures thereof.

10. The disposable hygiene article of claim 1, wherein the microfibers have an average diameter of 10 to 30 μm; the densely packed nanofibers and the loosely packed nanofibers have an average diameter of 100 to 1,000 nm; the plurality of pores has a pore-to-pore distance of 100-1,000 μm; the densely packed nanofibers and the loosely packed nanofibers comprise a polymer selected from the group consisting of polymers comprising cellulose acetate (CA), polyamide 6 (PA6), polystyrene (PS), polyacrylonitrile (PAN), polyacrylonitrile co-polymer with methyl acrylate (n-PAN), polyvinylpyrrolidone (PVP), polyvinyl alcohol (PVA), poly(lactic acid) (PLA), poly(lactic-co-glycolic acid) (PLGA), polybutylene terephthalate (PBT), polyurethane (PU), gelatin, chitosan, polyhydroxybutyrate-co-hydroxyvalerate (PHBV), and combinations thereof; and the top sheet comprises non-woven material comprising fibers comprising polyethylene, polypropylene, or combinations thereof.

11. The disposable hygiene article of claim 10, wherein the nanofiber layer further comprises an active ingredient selected from the group consisting of antioxidant, an anti-inflammatory, an anti-microbial, an emollient, and mixtures thereof.

12. The disposable hygiene article of claim 1 further comprising:
a back sheet; and
an absorbent core disposed between the back sheet and the nanofiber layer, wherein the absorbent core comprises a superabsorbent polymer (SAP).

13. The disposable hygiene article of claim 12, wherein the back sheet comprises polypropylene, polyethylene, nylon, polyester, or a combination thereof.

14. The disposable hygiene article of claim 12, wherein the SAP comprises sodium polyacrylate.

15. The disposable hygiene article of claim 12, wherein the microfibers have an average diameter of 10 to 30 μm; the densely packed nanofibers and the loosely packed nanofibers have an average diameter of 100 to 1,000 nm; the plurality of pores has a pore-to-pore distance of 100-1,000 μm; the densely packed nanofibers and the loosely packed nanofibers comprise a polymer selected from the group consisting of polymers comprising cellulose acetate (CA), polyamide 6 (PA6), polystyrene (PS), polyacrylonitrile (PAN), polyacrylonitrile co-polymer with methyl acrylate (n-PAN), polyvinylpyrrolidone (PVP), polyvinyl alcohol (PVA), poly(lactic acid) (PLA), poly(lactic-co-glycolic acid) (PLGA), polybutylene terephthalate (PBT), polyurethane (PU), gelatin, chitosan, polyhydroxybutyrate-co-hydroxyvalerate (PHBV), and combinations thereof; and the top sheet comprises non-woven material comprising fibers comprising polyethylene, polypropylene, or combinations thereof.

16. The disposable hygiene article of claim 15, wherein the nanofiber layer further comprises an active ingredient selected from the group consisting of an antioxidant, an anti-inflammatory, an anti-microbial, an emollient, and mixtures thereof.

17. The disposable hygiene article of claim 16, wherein the active ingredient is selected from the group consisting of calamine, dimethicone, kaolin, lanolin, petrolatum, talc, cornstarch, white petrolatum, zinc oxide, silver, copper, copper oxide, titanium oxide, iodine, triclosan, polyethylene glycol (PEG), sodium alginate, and mixtures thereof.

18. A diaper comprising the disposable hygiene article of claim 1.

19. A diaper comprising the disposable hygiene article of claim 15.
